(19) United States
(12) Patent Application Publication
(10) Pub. No.: US 2009/0075888 A1
(43) Pub. Date: Mar. 19, 2009

(54) Cardiovascular and Brain Cell Therapy Using Intracellular Ryanodine Receptor Modulation by the Estrogen Receptor Beta

(75) Inventors: Peter Koulen, Benbrook, TX (US); Volodymyr Rybalchenko, Keller, TX (US)

Correspondence Address: CHALKER FLORES, LLP, 2711 LBJ FRWY, Suite 1036, DALLAS, TX, 75234, US

(73) Assignee: University of North Texas Health Science Center at Fort Worth, Fort Worth, TX (US)

(21) Appl. No.: 12/205,862

(22) Filed: Sep. 6, 2008

Related U.S. Application Data
(60) Provisional application No. 60/972,176, filed on Sep. 13, 2007.

(51) Int. Cl. A61K 38/22; G01N 33/567; C12Q 1/70; C12N 5/02; A61P 25/00; A61P 9/00; A61K 31/56; C12Q 1/68

(52) U.S. Cl. 514/12; 435/7.21; 435/5; 435/6; 514/169; 435/375

(57) ABSTRACT

The present invention includes compositions and methods for screening for a candidate substance with ryanodine receptor (RyR)-modulatory activity, the method including: determining the ion-conducting ability and ability to change the concentration of the free cytoplasmic intracellular Ca²⁺ by the RyR modulated by Estrogen receptor-β (ERβ) in cells or cell membranes expressing RyR and ERβ combination with, or in the absence of the estrogen; contacting the cells or cell membranes with a candidate substance capable of modulation the interaction between RyR and ERβ; and measuring the RyR mediated ion-conducting ability of the cells or cell membranes to change the concentration of the free cytoplasmic intracellular Ca²⁺ by the candidate substance, whereby the modulatory activity of the candidate substance on RyR/ERβ interaction is determined.

### CROSS-REFERENCE TO RELATED APPLICATIONS

This application claims priority to U.S. Provisional Application Ser. No. 60/972,176, filed Sep. 13, 2007, the entire contents of which are incorporated herein by reference.

### TECHNICAL FIELD OF THE INVENTION

The present invention relates in general to the field of therapeutic uses for novel modulators of the ryanodine receptor.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with ryanodine receptors.

U.S. Pat. No. 6,462,066, issued to Mangat, et al., is directed to methods and compositions for treatment of ischemic neuronal reperfusion injury. Briefly, compositions and methods are disclosed for treatment of, or protection from, neuropathy resulting from reperfusion injury upon reversal of an ischemic condition, comprising treatment or prophylactic treatment of the patient with an antagonist of the type 3 ryanodine receptor, such that a rise in cytosolic Ca²⁺ concentration is prevented. Therapeutic compositions containing dantrolene or aminodantrolene are administered to the patient to prevent a rise in cytosolic Ca²⁺ that would otherwise result in Ca²⁺-mediated neuronal damage. Treatment of ischemic optic neuropathy by this method is shown, and the methods and compositions presented are also applicable to other ischemic reperfusion neuropathies, such as stroke, reperfusion injury after TPA treatment/carotid endarterectomy, seizures, and excitotoxic retinal damage in glaucoma.

United States Patent Application No. 20070196856, filed by Dong; Cun-Jian, et al., is directed to methods of determining activity of ryanodine receptor modulators. Briefly, methods are taught for identifying modulators of ryanodine receptors. In one embodiment the activity of the ryanodine receptor is stimulated to a baseline level and the ability of a test compound to increase or decrease the baseline level indicates that the test compound is a modulator of ryanodine receptor activity. The application includes a method for determining the ability of a test substance to modulate the activity of a ryanodine receptor (RyR) isoform, the method comprising: contacting a RyR isoform in a cell with an effective amount of a ryanodine receptor activating component and a test substance; and monitoring the release of Ca⁺⁺ by the RyR isoform.

In United States Patent Application No. 20070049630, Dong; Cun-Jian, et al., teach a method of using ryanodine receptor antagonists to treat amyotrophic lateral sclerosis. Briefly, a method of providing neural protection in human patients suffering from amyotrophic lateral sclerosis includes administering to the patients suffering from said amyotrophic lateral sclerosis an effective amount of a compound that is a ryanodine receptor antagonist in pharmaceutically acceptable vehicle to inhibit or prevent neuronal injury or death.

Finally, United States Patent Application No. 20060293266, filed by Marks; Andrew R., teaches the use of a phosphodiesterase 4D in the ryanodine receptor complex protects against heart failure. Briefly, Marks teaches compositions, methods and kits useful for treating and preventing ryanodine receptor associated disorders that include a PDE-associated agent and a pharmaceutically acceptable carrier. The present invention also provides methods for treating or preventing ryanodine receptor associated disorders including cardiac disorders and diseases, skeletal muscular disorders and diseases, cognitive disorders and diseases malignant hyperthermia, diabetes and sudden infant death syndrome.

### SUMMARY OF THE INVENTION

The present invention includes methods of screening for a candidate substance with ryanodine receptor (RyR)-modulatory activity, the method by determining the ion-conducting ability and ability to change the concentration of the free cytoplasmic intracellular Ca²⁺ by the RyR modulated by Estrogen receptor-β (ERβ) in cells or cell membranes expressing RyR and ERβ in combination with, or in the absence of estrogens; contacting the cells or cell membranes with a candidate substance capable of modulation the interaction between RyR and ERβ; and measuring the RyR mediated ion-conducting ability of the cells or cell membranes to change the concentration of the free cytoplasmic intracellular Ca²⁺ by the candidate substance, whereby the modulatory activity of the candidate substance on RyR/ERβ interaction is determined. In one aspect, the RyR-expressing cells are primary brain, cardiac and vascular tissues or primary cell cultures, cells transfected with a RyR receptor or cell lines that express the RyR receptor. In another aspect, the cell membranes may be bilayer lipid membranes (BLM), or Ca²⁺ release reagents (liposomes and microsomes). The amount of internalized ERβ for use with the present invention may be, e.g., between 1 μM to 100 μM.

In another aspect, the RyR ion-conducting ability and ability to influence the concentration of the free cytoplasmic intracellular Ca²⁺ are measured electrophysiologically, fluorescently or calorimetrically. The candidate substance may be an estrogen (estradiol, 17β-estradiol, E2, estriol, estrone) or a functional derivative, precursor, prodrug, homologue, analogue or salt thereof. Other examples of candidate substance(s) include an ERβ-specific binding agent including but not restricted to small molecules, peptides and proteins and is selected from a small molecule library. In one specific formulation of the present invention the candidate substance is not-internalizable. Alternatively, the candidate substance is an ERβ-specific binding agent delivered into the cell by gene transfer, peptide or protein delivery constructs comprising at least a portion of the ERβ. Additional examples of candidate substances include a plasmid, cosmid, artificial chromosome, viroid, virus and virus-like particles, nanoparticle and electrical, magnetic or chemical delivery reagents that deliver nucleic acids that express peptides or proteins comprising at least a portion of the ERβ into cells.

In another embodiment, the present invention includes a method of treatment of cardiac or vascular dysfunction in a human or animal subject comprising administering or intracellular synthesis of an effective amount of a low dose of an ERβ, ERβ fragment or derivative, ERβ-specific binding agent, including estrogen and other hormones acting through ERβ, for a time and under conditions sufficient for correction of cardiac and vascular contraction/relaxation to occur thereby rectifying said cardiac and vascular dysfunction or pathology. The amount of the ERβ-specific binding agent may be modulated based on the effect of the ERβ-specific binding agent on the RyR obtained from the subject measured by RyR ion conducting ability, Ca²⁺-induced Ca²⁺ release (CICR) or both. The cardiac dysfunction may be a myocardial contractile failure, ischemic heart disease, systemic inflammatory states such as sepsis, cardiac hypertrophy (calcium overload), cardiomyopathy such as arrhythmogenic right ventricular dysplasia type-2 (ARVD2), and drug (e.g. cocaine)-induced cardiomyopathy, infarction, dysrhythmia, congestive heart failure, or heart attack.

In another embodiment, the present invention includes a dosage form that includes a low dose estrogen or candidate substance sufficient to treat a cardiovascular disease, wherein the dosage form is adapted to provide intracellular content of an estrogen or candidate substance that modulate the ERβ receptor based on the level of membrane ryanodine receptor (RyR) activity measured as ion-conducting ability of the RyR or Ca²⁺-induced Ca²⁺ release (CICR) from the endoplasmic reticulum of the cardiac vascular or neuronal tissue or primary cell culture in vitro. The cardiac or vascular dysfunction may be a myocardial contractile failure, ischemic heart disease, systemic inflammatory states such as sepsis, cardiac hypertrophy (calcium overload), cardiomyopathy such as arrhythmogenic right ventricular dysplasia type-2 (ARVD2), and drug-induced (e.g. cocaine) cardiomyopathy, infarction, dysrhythmia, congestive heart failure, or heart attack. The dosage form may be adapted for patients suffering from a loss of estrogen that is caused iatrogenically, by ovariectomy, by menopause, or due to normal aging. In another embodiment, the low dose estrogen or candidate substance crosses the blood-brain barrier and/or is dissolved in a lipophilic pharmacophor and is suitable for intravenous injection, parenteral administration or oral administration and is administered one or more times daily over a predetermined period.

Another embodiment of the present invention includes a method of treatment of neuronal dysfunction in a human or animal subject by administering or intracellular synthesis of an effective amount of a low dose of an ERβ, ERβ fragment or derivative, ERβ-specific binding agent, including estrogen and other hormones acting through ERβ, for a time and under conditions sufficient for reduced neurodegeneration, increased generation, mobility or interconnectivity of the neurons and other brain cells to occur, thereby rectifying said neuronal or brain dysfunction or pathology. The neuronal or brain dysfunction may be selected from the group consisting of schizophrenia, minimal brain dysfunction, mania, Alzheimer's disease, attention deficit disorder (ADD), obsessive-compulsive disorder (OCD), learning deficit, dysmnesia, agnosia, amnesia and apraxia, Parkinsonism and its iatrogenic forms, Huntington's disease, glaucoma, macular degeneration, retinitis pigmentosa and acute diseases of the central nervous system (stroke, ischemia). In a related embodiment, a dosage form is prepared that includes a low dose estrogen or candidate substance sufficient to treat a neuronal dysfunction, wherein the dosage form is adapted to provide intracellular content of an estrogen or candidate substance that modulate the ERβ receptor based on the level of membrane ryanodine receptor (RyR) activity measured as ion-conducting ability of the RyR or Ca²⁺-induced Ca²⁺ release (CICR) from the endoplasmic reticulum of the cardiac vascular or neuronal tissue or primary cell culture in vitro. The dosage form may be adapted to treat a neuronal or brain dysfunction is selected from the group consisting of schizophrenia, minimal brain dysfunction, mania, Alzheimer's disease, attention deficit disorder (ADD), obsessive-compulsive disorder (OCD), learning deficit, dysmnesia, agnosia, amnesia and apraxia, Parkinsonism and its iatrogenic forms, Huntington's disease, glaucoma, macular degeneration, retinitis pigmentosa and acute diseases of the central nervous system (stroke, ischemia).

### BRIEF DESCRIPTION OF THE DRAWINGS

For a more complete understanding of the features and advantages of the present invention, reference is now made to the detailed description of the invention along with the accompanying figures and in which:

FIG. 1 shows that ERβ activates RyR type 2 (RyR2).

FIG. 2 shows that ERβ activates “silent” RyR2.

FIG. 3 shows that RyR activated by the purified recombinant ERβ is dose-dependently and reversibly inhibited by subsequent E2 application.

FIG. 4 shows that ERβ at a concentration of 20 nM activates RyR2.

FIG. 5 shows that ERβ applied at 20 nM changes the biophysical parameters of RyR2.

FIG. 6 shows that ERβ at concentration of 10 nM activates RyR2.

FIG. 7 shows that ERβ applied at 10 nM changes the biophysical parameters of RyR2.

FIG. 8 shows the colocalization of ERβ.

FIG. 9 shows the effects of ERβ applied at low nanomolar concentrations shifts the pattern of the RyR channel openings to higher sublevels.

FIG. 10 shows the biphasic temporal effects on the RyR single channel current characteristics produced by ERβ application.

FIG. 11 shows that higher ERβ concentrations stimulate RyR stable sublevel openings.

FIG. 12 shows that ERβ dose-dependently increases the probability of higher RyR open sublevels.

FIG. 13 shows that ERβ and Ca²⁺ increase the RyR single channel activity in a synergistic way.

FIG. 14 demonstrates the activating effect of ERb does not prevent the RyR desensitization by high calcium concentrations.

FIG. 15 shows that RyR2 and ERβ are co-localized in cytoplasmic compartments of neuronal HT-22 cells.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “candidate substance” or “candidate compound” refers to any molecule that may potentially inhibit or activate the expression or activity of the ryanodine receptor (RyR) and RyR/ERβ interaction. A ryanodine receptor (RyR) activity modulator may be a compound that overall affects the interaction of RyR with Estrogen receptor-β (ERβ); or the activity of the RyR mediated by ERβ, e.g., ion-conducting ability of the RyR or Ca²⁺-induced Ca²⁺ release (CICR). As used herein “RyR” includes all isoforms, including all three known types and their isoforms and modifications thereof. Such an RyR/ERβ interaction modulator may also regulate RyR expression, translocation or transport, function, post-translational modification, location, or regulate more directly by preventing or promoting its activity, such as by binding ERβ or vice versa. Any compound or molecule described in the methods and compositions herein may be a RyR/ERβ modulator whether altering the RyR or the ERβ portion of the interaction. As used herein, ERβ refers to its long form and all naturally occurring isoforms or recombinantly generated modifications. As used herein, “estrogens” refers to estrogen and its derivatives, including estradiol, 17β-estradiol, estriol and estrone.

The candidate substance may be a protein or fragment thereof, a small molecule, or even a nucleic acid molecule. It may prove to be the case that the most useful pharmacological compounds will be compounds that are structurally related to RyR or that bind RyR. Using lead compounds to help develop improved compounds is known as “rational drug design” and includes not only comparisons with known modulators, but predictions relating to the structure of target molecules.

Candidate substances, compounds or modulators of the present invention will likely function to regulate i.e., inhibit, decrease, or activate, increase the expression or activity of RyR in a cardiac, vascular or neural cell. Such candidate substances may be inhibitors, or activators of ERβ. These candidate compounds may be antisense molecules, ribozymes, antibodies (including single chain antibodies), or organopharmaceuticals, but are not limited to such.

The present invention also provides methods for developing drugs that modulate RyR activity caused by RyR/ERβ interaction or variable expression that may be used to treat cardiac, vascular or neural diseases or conditions. One such method involves the prediction of the three-dimensional structure of a validated RyR/ERβ interaction modulator target using molecular modeling and computer stimulations. The resulting structure is then used in docking studies to identify potential small molecule inhibitors that bind in the enzyme's active site with favorable binding energies. Modulators identified may then be tested in biochemical assays to further identify RyR drug targets that alter the ion-conducting ability of RyR or Ca²⁺-induced Ca²⁺ release (CICR). The RyR modulators may then be evaluated for reduced neurodegeneration, increased generation, mobility or interconnectivity of the neurons and other brain cells to occur, thereby rectifying said neuronal or brain dysfunction or pathology. The neuronal or brain dysfunction may be selected from the group consisting of schizophrenia, minimal brain dysfunction, mania, Alzheimer's disease, attention deficit disorder (ADD), obsessive-compulsive disorder (OCD), learning deficit, dysmnesia, agnosia, amnesia and apraxia, Parkinsonism and its iatrogenic forms, Huntington's disease, glaucoma, macular degeneration, retinitis pigmentosa and acute diseases of the central nervous system (stroke, ischemia). The RyR modulators may then be evaluated for treatment of cardiac dysfunction, e.g., myocardial contractile failure, ischemic heart disease, systemic inflammatory states such as sepsis, cardiac hypertrophy (calcium overload), cardiomyopathy such as arrhythmogenic right ventricular dysplasia type-2 (ARVD2), and drug (e.g. cocaine)-induced cardiomyopathy, infarction, dysrhythmia, congestive heart failure, or heart attack.

Rational drug design is therefore used to produce structural analogs of substrates for RyR and/or specificity for ERβ. By creating such analogs, it is possible to fashion drugs which are more active or stable than the natural molecules, which have different susceptibility to alteration or which may affect the function of various other molecules. In one approach, one would generate a three-dimensional structure for the RyR targets of the invention or a fragment thereof. This could be accomplished by X-ray crystallography, computer modeling or by a combination of both approaches.

It also is possible to use antibodies to ascertain the structure of a target compound modulator. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Alternatively, small molecule libraries are available from commercial sources that are selected to meet the basic criteria for useful drugs in an effort to “brute force” the identification of useful compounds. Screening of such libraries, including combinatorially generated libraries (e.g., peptide libraries), is a rapid and efficient way to screen a large number of related (and unrelated) compounds for activity. Combinatorial approaches also lend themselves to rapid evolution of potential drugs by the creation of second, third and fourth generation compounds modeled from the active candidate substances and redesigned using the rational drug design methods described hereinabove.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. The pharmaceutical agents screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable compounds with RyR binding and/or modulating activity include antisense molecules, ribozymes and antibodies (or fragments thereof), specific for the target molecule. Such compounds are described in greater detail elsewhere in this document. For example, an antisense molecule that bound to a translational or transcriptional start site, or splice junctions, would be ideal candidate modulators.

In addition to the activating and inhibiting compounds initially identified, the inventors also contemplate that other sterically similar compounds may be formulated to mimic the key portions of the structure of the RyR modulators. Such compounds include peptidomimetics of peptide modulators, may be used in the same manner as the initial modulators.

A modulator according to the present invention may be one which exerts its inhibitory or activating effect upstream, downstream or directly on RyRs. Regardless of the type of modulator identified by the present screening methods, the effect of the inhibition or activation by such a compound results in the regulation in RyR activity or expression as compared to that observed in the absence of the added candidate substance, e.g., RyR ion conducting ability, Ca²⁺-induced Ca²⁺ release (CICR), RyR-ERβ binding or combinations thereof.

As used herein, the term “drug” refers to a chemical entity, whether in the solid, liquid, or gaseous phase which is capable of providing a desired therapeutic effect when administered to a subject. The term “drug” includes synthetic compounds, natural products and macromolecular entities such as polypeptides, polynucleotides, or lipids and also small entities such as neurotransmitters, ligands, hormones or elemental compounds. The term “drug” also refers to compounds whether it is in a crude mixture, as an extract, elixir, mixture or purified and isolated.

As used herein, “pharmaceutical or pharmacologically acceptable” refers to molecular entities and compositions that do not produce an adverse, allergic or other untoward reaction when administered to an animal, such as, for example, a human, as appropriate. Pharmaceutical compositions of the present invention include an effective amount of one or more modulators that inhibit or activate RyR expression or activity, and/or additional agents, dissolved or dispersed in a pharmaceutically acceptable carrier to a subject. The preparation of a pharmaceutical composition that contains at least one RyR or ERβ modulator or additional active ingredient will be known to those of skill in the art in light of the present disclosure, and as exemplified by Remington: The Science and Practice of Pharmacy, 21st Edition (2005) Lippincott Williams & Wilkins, relevant portions incorporated herein by reference. Moreover, for animal or human administration, it will be understood that preparations should meet sterility, pyrogenicity, general safety and purity standards as required by FDA Office of Biological Standards.

As used herein, “pharmaceutically acceptable carrier” refers to any and all salts, preservatives, drugs, drug stabilizers, gels, binders, excipients, disintegration agents, lubricants, sweetening agents, flavoring agents, dyes, solvents, dispersion media, coatings, surfactants, antioxidants, preservatives (e.g., antibacterial agents, antifungal agents), isotonic agents, absorption delaying agents, such like materials and combinations thereof, as would be known to one of ordinary skill in the art (see, for example, Remington: The Science and Practice of Pharmacy, 21st Edition (2005) Lippincott Williams & Wilkins, relevant portions incorporated herein by reference). Except insofar as any conventional carrier is incompatible with the active ingredient, its use in the therapeutic or pharmaceutical compositions is contemplated.

A pharmaceutical composition of the present invention may include different types of carriers depending on whether it is to be administered in solid, liquid or aerosol form, and whether it needs to be sterile for such routes of administration as injection. A pharmaceutical composition of the present invention can be administered intravenously, intradermally, intraarterially, intraperitoneally, intraarticularly, intrapleurally, intranasally, topically, intramuscularly, intraperitoneally, subcutaneously, subconjunctival, intravesicularlly, mucosally, intrapericardially, intraumbilically, orally, topically, locally, by inhalation (e.g., aerosol inhalation), by injection, by infusion, by continuous infusion, via a catheter, via a lavage, in lipid compositions (e.g., liposomes), or by other method or any combination of the forgoing as would be known to one of ordinary skill in the art (see, for example, see, for example, Remington: The Science and Practice of Pharmacy, 21st Edition (2005) Lippincott Williams & Wilkins).

The actual dosage amount of a composition of the present invention administered to a subject can be determined by physical and physiological factors such as body weight, severity of condition, the type of disease being treated, previous or concurrent therapeutic interventions, idiopathy of the patient and on the route of administration. The number of doses and the period of time over which the dose may be given may vary. The practitioner responsible for administration will, in any event, determine the concentration of active ingredient(s) in a composition and appropriate dose(s), as well as the length of time for administration for the individual subject. For example, the dosage form will generally provide an intracellular content of an estrogen or candidate substance that modulate the ERβ receptor based on the level of membrane ryanodine receptor (RyR) activity measured as ion-conducting ability of the RyR or Ca²⁺-induced Ca²⁺ release (CICR). At or about the cell, the amount of internalized ERβ or ERβ ligand for use with the present invention may be, e.g., between 1 pM to 100 μM.

In certain embodiments, pharmaceutical compositions may include, for example, at least about 0.1% of an active compound. In other embodiments, the active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. Other non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

The composition may also include various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The RyR and/or ERβ modulator(s) may be formulated into a composition in a free base, neutral or salt form. Pharmaceutically acceptable salts, include the acid addition salts, e.g., those formed with inorganic acids such as for example, hydrochloric or phosphoric acids, or such organic acids as acetic, oxalic, tartaric, mandelic or other acids. Other salts include those formed with the free amino groups of a proteinaceous compositions. Salts formed with free carboxyl groups can also be derived from inorganic bases such as for example, sodium, potassium, ammonium, calcium or ferric hydroxides; or such organic bases as isopropylamine, trimethylamine, histidine, procaine or others.

Liquid dosage forms may be formulated in which a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

The RyR and/or ERβ modulator(s) may also be prepared for oral administration. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Carriers for oral administration may also include inert diluents, assimilable edible carriers or combinations thereof. The oral composition may be prepared as a syrup or elixir. A syrup or elixir, and may comprise, for example, at least one active agent, a sweetening agent, a preservative, a flavoring agent, a dye, a preservative, or combinations thereof. Oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof, an excipient, such as, for example, dicalcium phosphate, mannitol, lactose, starch, magnesium stearate, sodium saccharine, cellulose, magnesium carbonate or combinations thereof, a disintegrating agent, such as, for example, corn starch, potato starch, alginic acid or combinations thereof, a lubricant, such as, for example, magnesium stearate; a sweetening agent, such as, for example, sucrose, lactose, saccharin or combinations thereof, a flavoring agent, such as, for example peppermint, oil of wintergreen, cherry flavoring, orange flavoring, etc.; or combinations of any of the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

The RyR and/or ERβ modulator(s) may also be prepared as a dry powder for resuspension or resuspended in a sterile injectable solution by incorporating the active compounds in the required amount followed by sterilization. Generally, dispersions are prepared by incorporating the sterilized active ingredient(s) into a sterile vehicle that includes the basic dispersion medium and/or the other ingredients. In the case of sterile powders used for the preparation of sterile injectable solutions, suspensions or emulsion, the preparation may be, e.g., vacuum- or freeze-dried to yield a powder of the active ingredient plus any additional desired ingredient from a previously sterile-filtered liquid medium thereof. The liquid medium may be suitably buffered and rendered isotonic prior to injection with sufficient saline or glucose. Highly concentrated compositions for direct injection may also be prepared, e.g., where the use of DMSO as solvent is envisioned to result in extremely rapid penetration, delivering high concentrations of the active agents to a small area.

Generally, the composition should be stable under common conditions of manufacture and storage, and preserved against the contaminating action of microorganisms, such as viruses, bacteria and fungi. It will be appreciated that endotoxin contamination should be kept minimally at a safe level, for example, less that 0.5 ng/mg protein.

Estrogen (E2, 17β-estradiol) is an efficient natural agent against heart and vascular cell ischemic necrosis and also serves as protective agent against cardiac cell hypertrophic transformation leading progressively to heart failure. The most obvious mechanism of E2 cardioprotection is thought to be a Ca²⁺-dependent increase in cellular NO. In various cell types E2 produces rapid transients in intracellular Ca²⁺ (Ca²⁺ᵢ) concentration potentially controlled by estrogen receptor beta (ERβ), but the effector protein(s) activated by the ERβ and participating in the Ca²⁺ᵢ concentration changes is unknown. One of the possible effectors of the ER_{β} might be the endoplasmic reticulum membrane ryanodine receptor (RyR), which controls the cytoplasmic Ca²⁺ᵢ concentration through Ca²⁺-induced Ca²⁺ release (CICR) from the endoplasmic/sarcoplasmic reticulum. The discovery of the mechanism underlying the functional regulation of the RyR by ERβ, will enable the development of pharmacological or molecular biological treatments of injured, intrinsically or extrinsically damaged or hypertrophic myocardial and vascular tissue through modulation of ERβ/RyR mediated Ca²⁺ᵢ signaling. These medical treatments could be used in conditions when natural E2 production by the organism is reduced, or alternatively, when systemic administration of chronic E2 doses might produce harmful side effects.

Using an electrophysiological approach, we have discovered, working with isolated RyR type 2 (same type as expressed in the cardiomyocytes) incorporated in lipid bilayers, that addition of the physiologically relevant low concentrations of recombinant ERβ (˜5 to 20 nM) to the cytoplasmic side of the receptor significantly increased single channel currents produced by openings of the RyR ion channel. The increase in the RyR channel activity by ERβ occurred in the presence of E2, as well as in its absence. This result means that there potentially exist three ways of ERβ-mediated modulation of the RyR-controlled Ca²⁺ᵢ release that can be used for the development of therapeutic protocols. First, enhanced production of endogenous ERβ in the cytoplasm would increase RyR sensitivity to its natural ligand Ca²⁺. Secondly, by applying either E2 (or another selective ERβ activator) or selective inhibitor of the ER_{β}, to modify the ability of ERβ to bind the RyR and exert modulating effects on RyR activity. Thirdly, by applying either E2 or another selective ERβ activator or selective inhibitor of the ER_{β} to the pre-existing complex includes RyR and the unliganded ERβ, to modulate activity of the RyR within the existing molecular complex.

Resulting control over Ca²⁺-dependent protective cellular mechanisms can be used to protect heart or vascular ischemic tissue against necrosis or apoptosis and/or to prevent hypertrophic transformation of the myocardium or vascular smooth muscle tissue produced by unfavorable conditions (hypertension, adrenergic hyperstimulation, etc.). Since the various types of RyRs (including the cardiac RyR type 2) are ubiquitously expressed in vascular, muscle cells and in neurons, as well as in other brain cells, the discovered RyR modulation by ERR can be used for cellular protection in pathologically modified blood vessel, muscle and brain tissues through the mechanisms analogous to those discussed in relation with cardioprotection.

Estrogen treatment has been, and is currently used as a component of hormone replacement therapy (HRT) in post-menopausal women to protect against osteoporosis and negative consequences of hormone deficiency in pre-menopausal women. However, multiple side-effects of prolonged HRT (increased risk of breast cancer and others) have been revealed in wide-scale clinical trials, suggesting that alternative more discriminative therapeutic strategies need to be developed instead of the bold increase of estrogen blood content. Our discovery anticipates targeting a specific type of ERs, namely ERβ and its multiple natural isoforms generated by alternative splicing, which are differentially expressed in various cells and organs and therefore can be targeted on the cell-specific basis. ERβ in various cell types is involved in rapid non-genomic responses, in contrary to the other major type, ERα, which is mostly involved in long-term genomic responses including the potential of contributing to carcinogenic transformations.

The present invention can be used to identify, isolate and optimize selective ligands of ERβ, which can control ERβ/RyR interaction without estrogen involvement. This approach reduces potential feminizing effects of high doses of estrogens and can be used both in women and men.

ER_{β}-regulating peptides as well as other molecular biologically mediated therapies can be developed instead of or in conjunction with systemically applied small molecule approaches, to selectively disrupt or activate the ER_{β}/RyR interaction in specific group of cells using local gene therapy approaches.

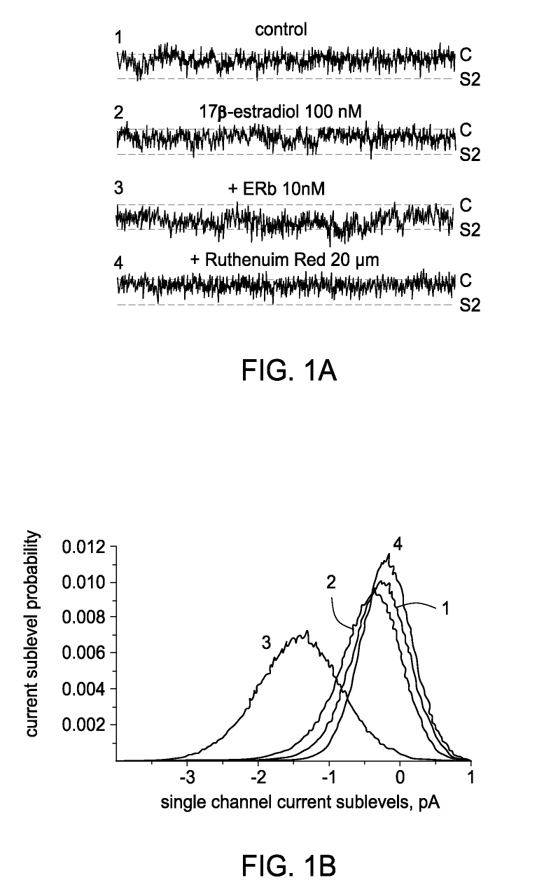

FIG. 1, A to D, summarizes the electrophysiological studies on the effects of ERβ, specifically the ERβ1 full length isoform, on RyR2. For the representative RyR channel shown in FIGS. 1A and 1B, the control excessive application of estrogen (17_{β}-estradiol, E2) (trace and histogram #2) did not produce significant effects, indicating the absence of endogenous ERs coupled with RyR2 in incorporated microsomal membrane fragment and of E2 on the channel itself. The application of soluble ER_{β} to RyR2 channels produced a significant increase in the amplitude of single channel currents (trace and histogram #3), which was subsequently blocked by the RyR2 channel blocker Ruthenium Red (trace #4). For the RyR presented in FIGS. 1A and 1B, the effect of ERβ was tested in the presence of E2. However, the statistically significant activating effect of ERβ on RyR2 was also observed in the absence of E2 (FIGS. 1C and 1D). Therefore, it is important to note that both, E2-bound and unliganded ER_{β} are capable of modulation of the RyR2 channel activity.

FIG. 1 shows that ERβ activates RyR type 2 (RyR2). FIG. 1A shows 2 s fragments of single-channel currents produced by mouse brain RyR2 (at pCa 7) incorporated in artificial lipid bilayer in control (1) and after subsequently added 100 nM E2 (2), 10 nM ERβ (3) and 20 μM Ruthenium Red (4). Dotted lines represent closed state (C) and −2 pA sublevel (S2). FIG. 1B shows histograms obtained from contiguous 60 s-long recordings from the same RyR2 and conditions presented in part A. FIG. 1C shows normalized and averaged amplitudes of mean single RyR2 channel currents obtained from six different receptors in control conditions and after addition of 10 nM of unliganded ERβ. FIG. 1D shows normalized and averaged values of RyR2 channel openings to S2 sublevel obtained from six different receptors in control conditions and after addition of 10 nM of unliganded ERβ. (* p<0.05).

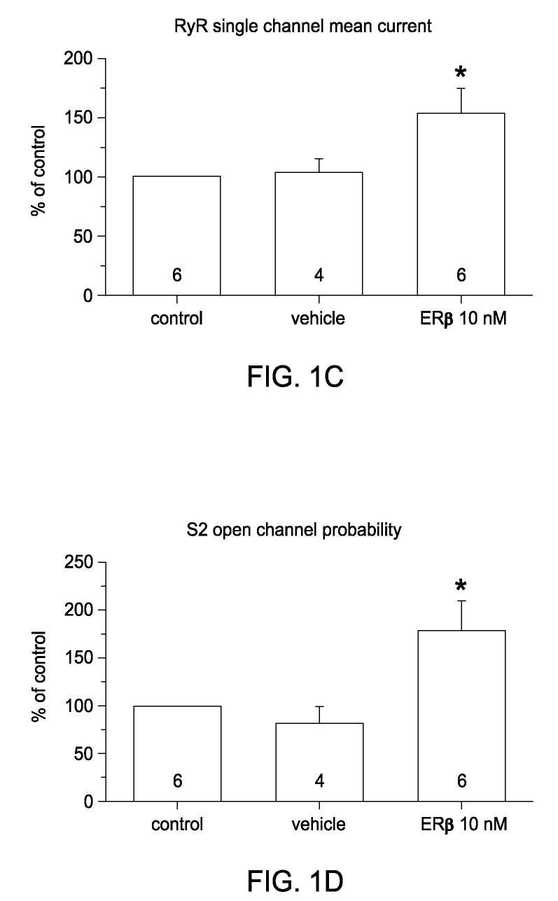

Another observed effect of ERβ on RyR single channel currents was the ability to activate a “silent” RyR inhibited by unidentified environmental factors (supposedly by the RyR inhibitory accessory protein FKBP). For the RyR presented in FIGS. 2A and 2B, for which no channel activity was initially observed at optimal activating Ca²⁺ concentration of 1 μM (FIG. 2A, trace #1), the addition of 5 nM ERβ produced channel openings to various sublevels approaching to a fully opened channel state of ˜−4 pA (trace #2). The transition from predominantly closed state to a large variety of open sublevels is reflected on histograms (FIG. 2B) calculated from continuous 60 s recordings in control vs. test conditions. These results demonstrate that the RyR channel ability of passing ionic currents through the endoplasmic reticulum membrane can be directly modulated by the ERβ.

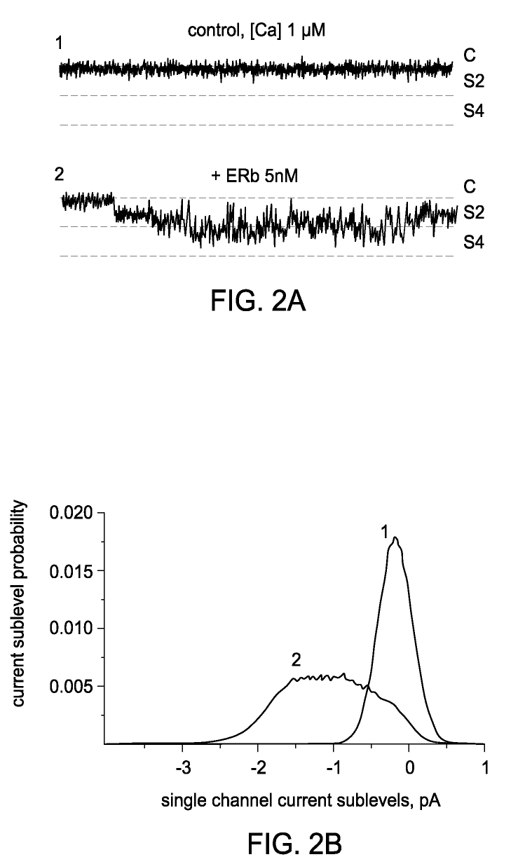

In addition to the activating effects of ERβ on RyR single channel currents in the absence of the ERβ-ligand, 17β-estradiol (estrogen, E2), RyR activity was measured in the presence of both ERβ and E2. FIG. 3 demonstrates that, while unliganded ERβ produces a consistent activation of RyR single channel currents, further addition of E2 reduces channel activity below baseline levels (FIG. 3, comparison of channel activity after addition of 50 and 100 nM E2 with baseline activity measured at pCa8) in a dose-dependent (FIG. 3, comparison of channel activity after addition of 50 and 100 nM E2, respectively) and reversible manner (FIG. 3, comparison of channel activity after addition of E2 with washout).

These results demonstrate that: (1) the dose-dependency indicates specificity and potential for disease—and dosing—specificity; (2) the reversible activation indicates specificity and potential for pharmacological applications; (3) effective block of RyR is achieved by E2-liganded ERβ (approximately 5-10 decrease); (4) the biological equivalents (˜ 1/100) of low doses of E2 used in the in vitro studies are highly physiologically relevant and indicate potential for pharmacological applications; and (5) absence of E2 can lead to higher than physiologically normal activity of RyR potentially contributing to calcium toxicity in E2 deprived systems such as in females after ovariectomy and after menopause as well as for both sexes during aging.

FIG. 2 shows that ER_{β} activates “silent” RyR2. FIG. 2A, 2 s fragments of single-channel currents produced by mouse RyR2 (at pCa 6) at control (1) and after addition of 5 nM ERβ (2). Dotted lines represent closed state (C), 2 pA sublevel (S2) and −4 pA sublevel (S4). FIG. 2B, histograms obtained from contiguous 60 s-long recordings from the same RyR2 and conditions presented in part A. The wide range of RyR single channel current sublevels appeared after the ER_{β} treatment (2 vs. 1).

FIG. 3 shows that RyR activated by the purified recombinant ERβ is dose-dependently and reversibly inhibited by subsequent E2 application. Mean single channel current is plotted over time and additions of protein and compounds are indicated as bars above the data points. pCa, negative decimal logarithm of the free calcium ion concentration; E2, 17β-estradiol.

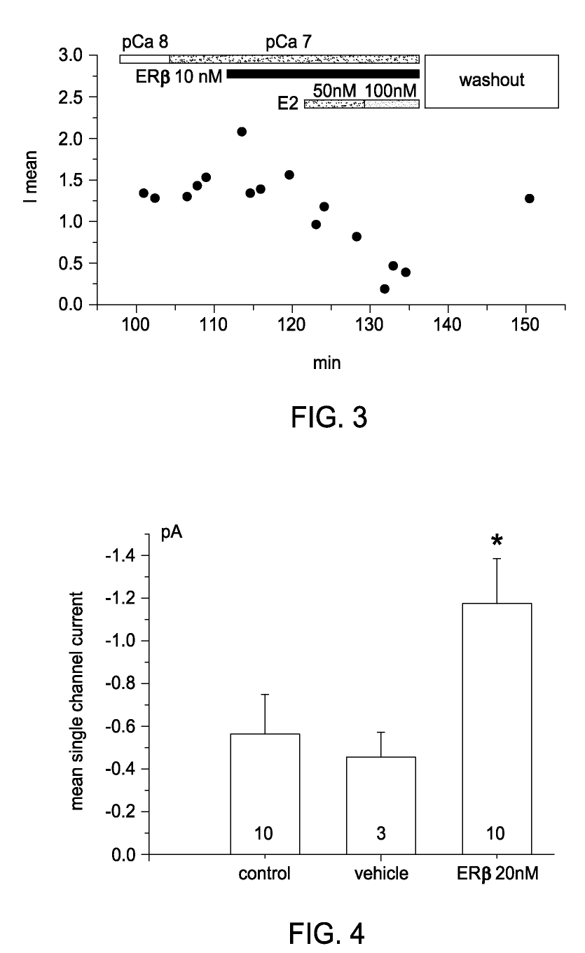

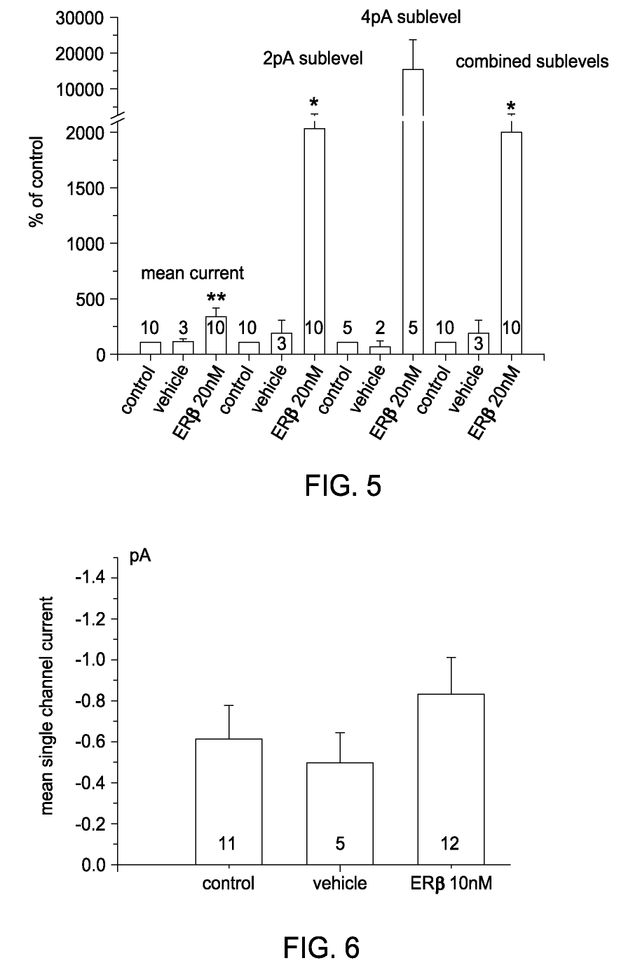

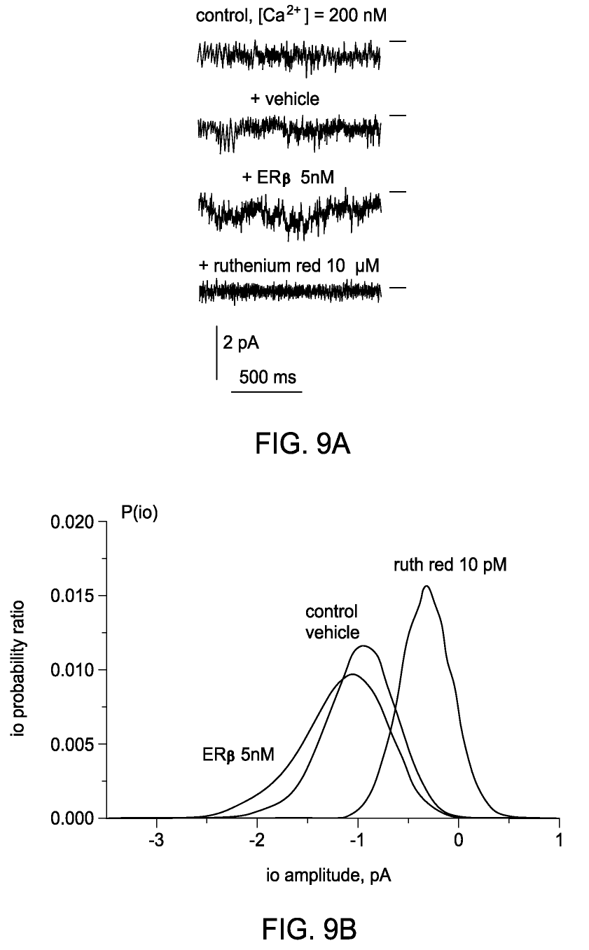

FIGS. 4-7 show the activating properties of ERβ on RyR single channel activity. The largest and most significant effect of ERβ on RyR2 channel activity was seen at the physiologically most relevant fully open state of the receptor (4 pA substate; FIGS. 5 and 7).

FIG. 4 shows that ERβ at a concentration of 20 nM activates RyR2. Averaged amplitudes of mean single RyR2 channel currents obtained in control conditions and after addition of 20 nM of unliganded ERβ. (* p<0.05).

FIG. 5 shows that ERβ applied at 20 nM changes the biophysical parameters of RyR2. Normalized and averaged amplitudes of mean single RyR2 channel currents, openings to the 2 pA sublevel and to the fully open state (4 pA sub-level) obtained in control conditions and after addition of 20 nM of unliganded ERβ are plotted as % of control. The largest and most significant effect of ERβ on RyR2 channel activity was seen at the physiologically most relevant fully open state of the receptor (4 pA substate) (* p<0.05, ** p<0.01).

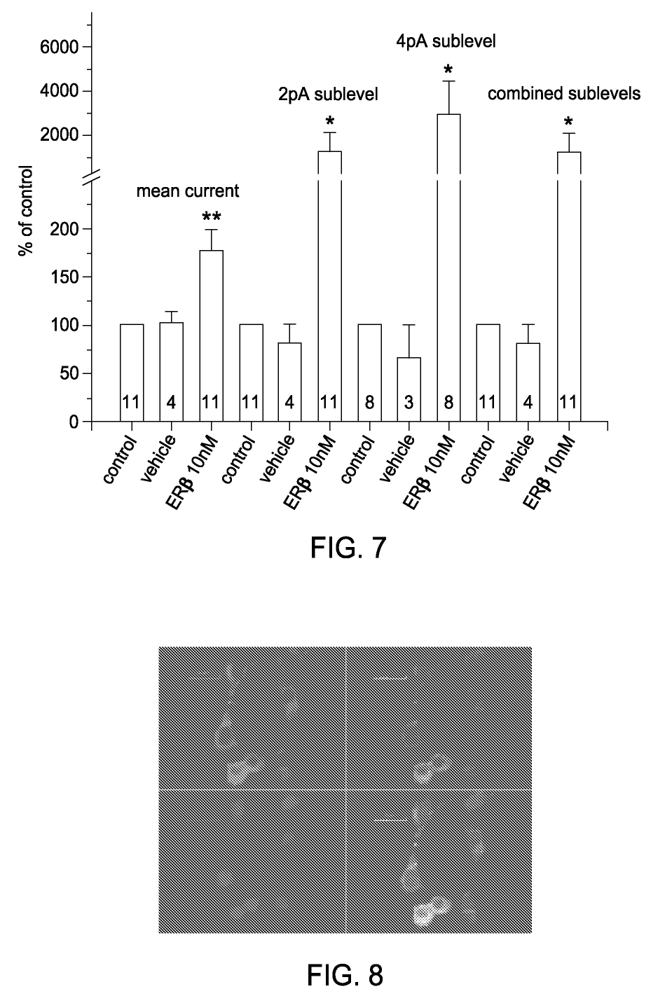

FIG. 6 shows that ERβ at a concentration of 10 nM activates RyR2. Averaged amplitudes of mean single RyR2 channel currents obtained in control conditions and after addition of 10 nM of unliganded ERβ.

FIG. 7 shows that ERβ applied at 10 nM changes the biophysical parameters of RyR2. Normalized and averaged amplitudes of mean single RyR2 channel currents, openings to the 2 pA sublevel and to the fully open state (4 pA sub-level) obtained in control conditions and after addition of 10 nM of unliganded ERβ are plotted as % of control. The largest and most significant effect of ERβ on RyR2 channel activity was seen at the physiologically most relevant fully open state of the receptor (4 pA substate) (* p<0.05, ** p<0.01).

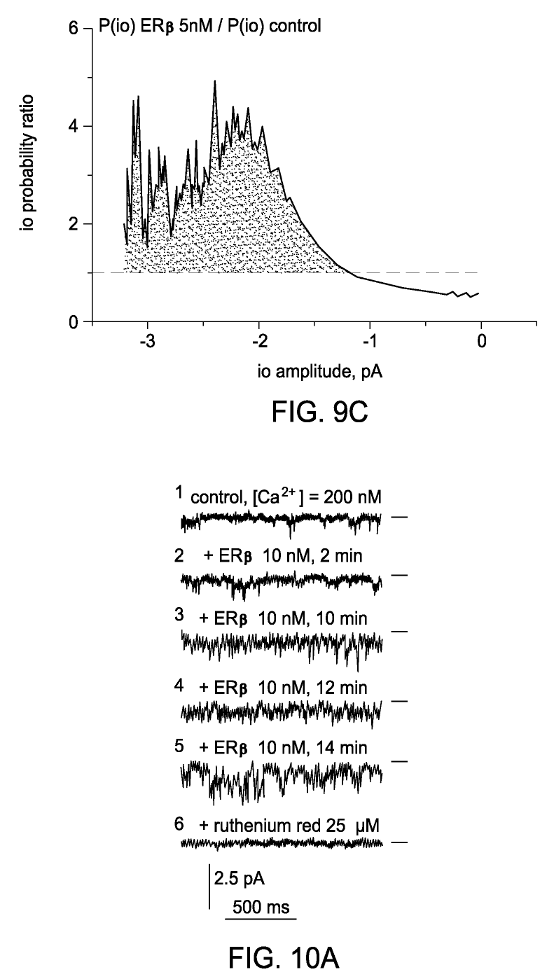

FIG. 8 shows the co-localization of ERβ (red; labeled with antibody ERβ H-150, Santa Cruz Biotechnology Inc., Santa Cruz, Calif. and for immunofluorescence detection with the secondary antibody Alexa 594-labeled goat anti-mouse IgG antibody (Invitrogen, Carlsbad, Calif.) and RyR2 (green; labeled with antibody MA3-916, clone C3-33, ABR—Affinity BioReagents, Inc., Golden, Colo. and for immunofluorescence detection with the secondary antibody Alexa 488-labeled goat anti-rabbit IgG) in the murine hippocampal cell line HT22. DNA was stained with DAPI (Invitrogen, Carlsbad, Calif.; blue label) to visualize nuclei. Scale bars, 25 μm.

It was found that the most pronounced effect occurs with the physiologically most relevant fully open state of the receptor (4 pA substate; FIGS. 5 and 7) indicating that the process in vitro is a true representation of effects occurring in vivo.

The physiological relevance of an involvement ERβ in calcium signaling indicates pharmacological applications related to E2 depletion (iatrogenic, ovariectomy, menopause, normal aging) and related to the need for control of intracellular calcium concentration (neurodegenerative diseases, cardiovascular disease).

In addition to the electrophysiological studies, ERβ and the cardiac RyR (RyR2) were colocalized in cultured cells using immunocytochemistry (FIG. 8). These results are relevant for the proposed application because they indicate the cell biological potential for protein-protein interaction underlying the modulation of RyR by liganded and unliganded ERβ providing a rationale for potential pharmacological applications.

These results demonstrate that the RyR channel ability of passing ionic currents through the endoplasmic reticulum membrane can be directly modulated by the ERβ and that E2 bound and E2 deficient ERβ have opposite effects on RyR activity providing the rationale for pharmacological applications related to E2 depletion (iatrogenic, ovariectomy, menopause, normal aging) and related to the need for control of intracellular calcium concentration (neurodegenerative diseases, cardiovascular disease).

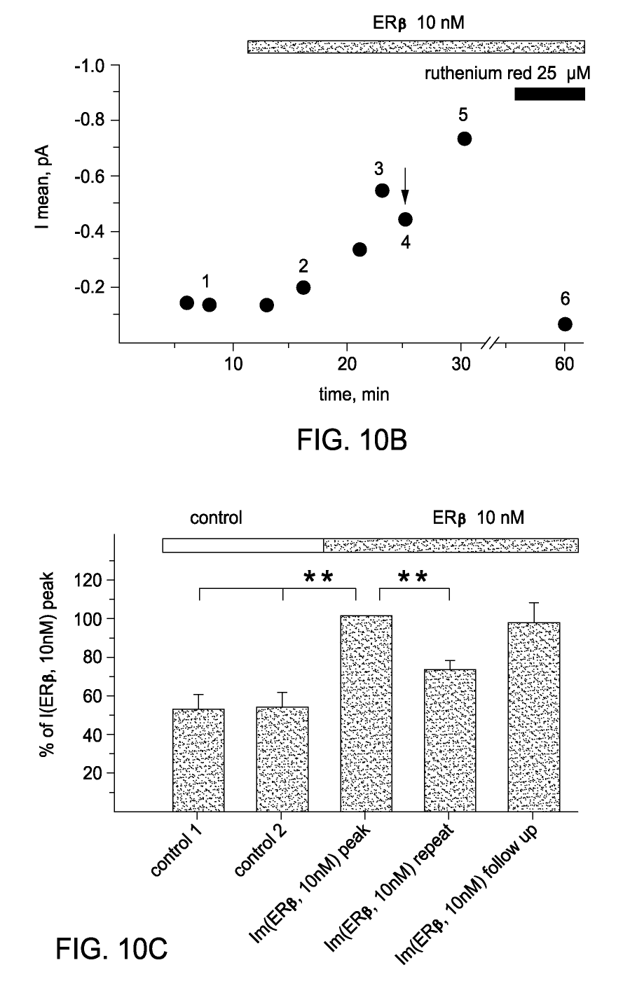

FIG. 9 shows the effects of ERβ applied at low nanomolar concentrations shifting the pattern of the RyR channel openings to higher sublevels. FIG. 9A shows representative 2 s long trace fragments of continuous single channel current recordings from the same RyR obtained at [Ca²⁺]_{cis}=200 nM in control conditions, after the ERβ vehicle solution, ERβ (5 nM) and RyR blocker ruthenium red (10 μM) application. RyR close state is indicated by horizontal line to the right of each trace. FIG. 9B shows the probability histograms for open channel current sublevels iₒ (P(iₒ)) calculated after expanded 60 s continuous current recording traces (comprising fragments from part 9A) at the same study conditions, which show a typically non-detectable influence of the vehicle solution and increased RyR channel openings to higher iₒ sublevels after the ERβ application. FIG. 9C shows the ratio of the P(iₒ) values calculated after and before the ERβ 5 nM application, obtained by numerical division of the corresponding histogram traces from part B, reflecting more than twice increase in P(iₒ) after the ERβ 5 nM treatment over the entire range of the meaningful RyR open sublevels.

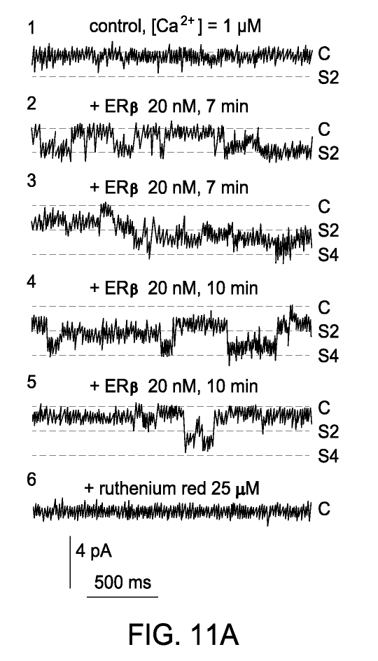

FIG. 10 shows the biphasic temporal effects on the RyR single channel current characteristics produced by ERβ application. FIG. 10A shows representative 2 s long trace fragments of continuous single channel current recordings from the same RyR obtained in chronological order at [Ca²⁺]_{cis}=200 nM in control conditions (1) and 2 min (2), 10 min (3), 12 min (4) and 14 min (5) after the ERβ (10 nM) application, followed by subsequent complete channel block with 25 μM of ruthenium red (6). FIG. 10B shows the time-course of the mean current (Iₘₑₐₙ) values calculated after expanded 60 s continuous recordings comprising fragments from part A obtained during progression of the ERβ application effect on the RyR presented in part FIG. 10A. Notable is a typical transient decrease in the Iₘₑₐₙ after the initial channel activation by ERβ (pointed by arrow), followed by stabilization of the activation effect. Numbers on the graph represent the time intervals corresponding to the fragments in part FIG. 10A. FIG. 10C shows the averaged Iₘₑₐₙ values obtained from 9 different RyRs during two control 60 s intervals preceding the ERβ 10 nM application (control 1 & 2), and during intervals of the initial increase (I_{mean, peak}), transient decrease (I_{mean, repeat}) and stabilization (I_{mean, follow-up}) of the RyR single channel current activated by 10 nM ERβ. Data are normalized by the Iₘₑₐₙ(ERβ, 10 nM)ₚₑₐₖ point corresponding to the initial RyR activation by ERβ. (** p<0.01).

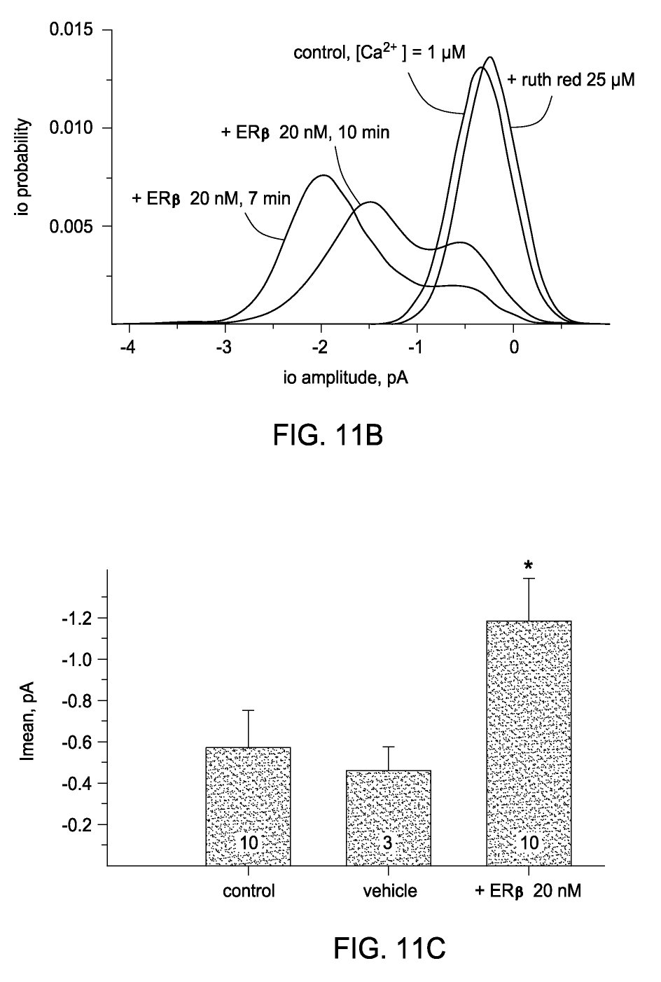

FIG. 11 shows that higher ERβ concentrations stimulate RyR stable sublevel openings. FIG. 11A shows representative 2 s long trace fragments of continuous single channel current recordings from the same low activity (silent) RyR obtained at [Ca²⁺]_{cis}=1 μM in control conditions (1) and after the ERβ 20 nM application for 7 min (2,3) and 10 min (4,5), followed subsequently by the ruthenium red (25 μM) block (6). Closed state (C) and conventional iₒ=2 pA (S2) and iₒ=4 pA (S4) sublevels are marked to the right of traces. FIG. 11B shows P(iₒ) histograms calculated after expanded 2 min continuous current recording traces (comprising fragments from part 11A) revealing an additional peak reflecting stable RyR openings at around the S2 sublevel after ERβ 20 nM treatment. (Shallowing and the rightward shift of the 10 min relative to 7 min histogram reflects the biphasic RyR activation by ERβ, see FIG. 10). FIG. 11C shows the averaged absolute values of Iₘₑₐₙ obtained after 60 s long RyR single channel continuous recordings in control conditions ([Ca²⁺]_{cis}=200 nM or 1 μM) and after application of the vehicle or ERβ 20 nM containing solutions. The number of the data points from different experiments taken for averaging is presented at the bottom of the columns. (* p<0.05).

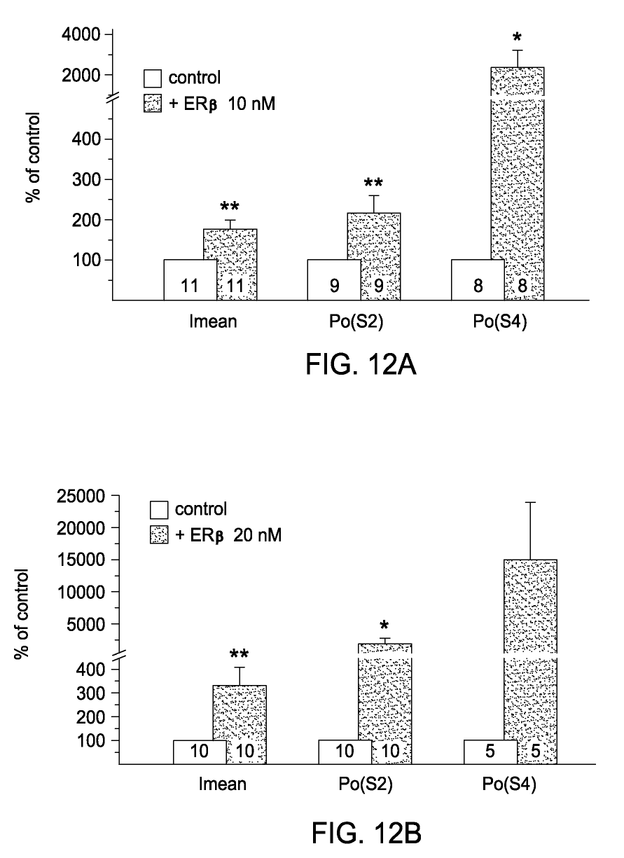

FIG. 12 shows that ERβ dose-dependently increases the probability of higher RyR open sublevels. The columns represent normalized to the control conditions ([Ca²⁺]_{cis}=200 nM or 1 μM) and averaged values of Iₘₑₐₙ and conventional open probabilities for −2 pA (Pₒ(S2)) and −4 pA (Pₒ(S4)) sublevels calculated for individual RyRs during the initial peak increase of the RyR channel activity by the ERβ applied at concentrations of 10 nM (top panel) and 20 nM (bottom panel). The number of averaged points is presented on the bottom of the columns. (** p<0.01, * p<0.05).

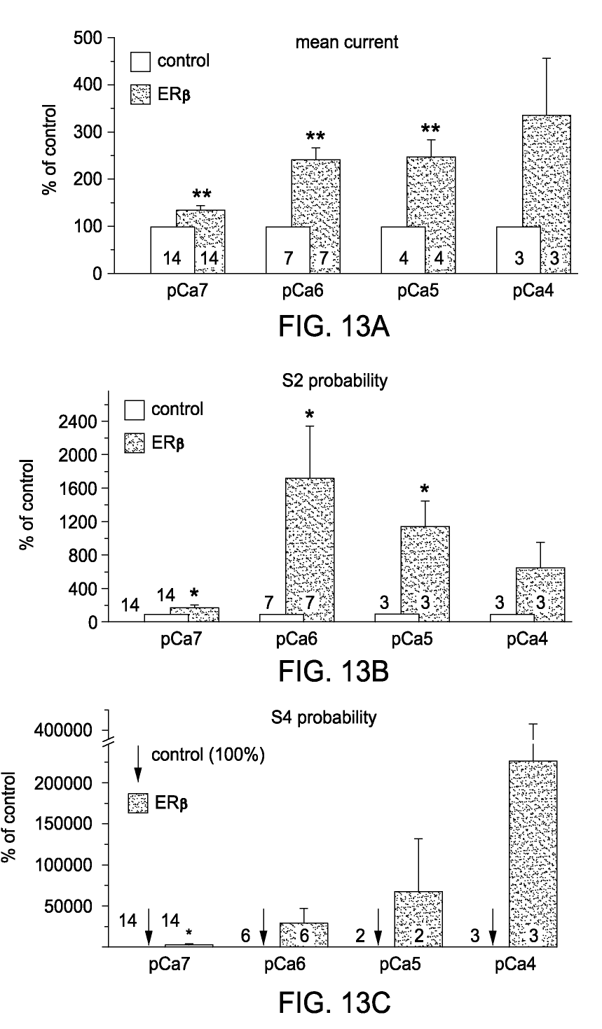

FIG. 13 shows that ERβ and Ca²⁺ increase the RyR single channel activity in a synergistic way. The columns represent normalized to the control conditions (before ERβ) averaged values of Iₘₑₐₙ (top), Pₒ(S2) (middle) and Pₒ(S4) (bottom) calculated for individual RyRs during the initial peak increase of the RyR channel activity by the ERβ application (10 or 20 nM) as function of [Ca²⁺]_{cis} concentrations (pCa 7, 6, 5 and 4) at which the ERβ was applied. Arrows at the bottom panel (S4 probability) indicate the indistinguishable control columns (100% level). The activating effect of the ERβ towards stimulation of the fully open S4 state of the RyR is more pronounced at higher [Ca²⁺]_{cis} (i.e. pCa 4). (** p<0.01, * p<0.05).

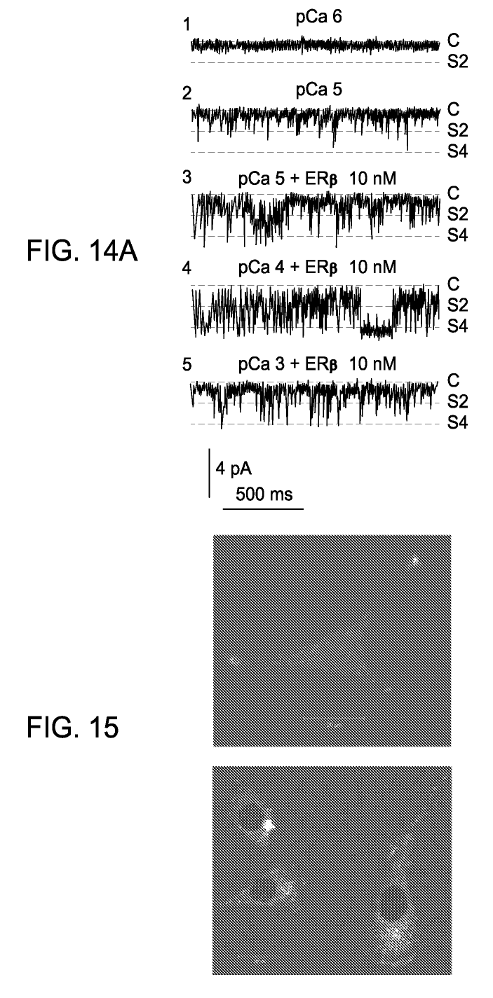

FIG. 14 demonstrates that activating effect of ERb does not prevent the RyR desensitization by high calcium concentrations. FIG. 14A shows representative 2 s long trace fragments of continuous single channel current recordings from the same RyR obtained at pCa 6 (1), pCa 5 before (2) and after (3) 10 nM ERβ application and at pCa 4 (4) and pCa 3 (5) at sustained presence of 10 nM ERβ. Closed state (C), iₒ=2 pA (S2) and iₒ=4 pA (S4) sublevels are marked to the right of traces. FIG. 14B shows the time-course presentation of the mean current (Iₘₑₐₙ) values calculated after expanded 60 s continuous recordings comprising fragments from part FIG. 14A. Bars on top indicate the time course of [Ca²⁺]_{cis} is changes and ERβ application. Notable is a sharp decrease in Iₘₑₐₙ values at pCa 3 due to the RyR desensitization after strong previous Iₘₑₐₙ increase at pCa 4 at the presence of 10 nM ERβ. Numbers on the graph represent the time intervals corresponding to the fragments in part FIG. 14A. FIG. 14C shows P(iₒ) histograms calculated after expanded 60 s continuous current recording traces (corresponding to numbered intervals from part FIG. 14B). A broad range of iₒ sublevels appearing at pCa4 (flattened line 4) decreased at pCa3 (line 5) due to the RyR desensitization.

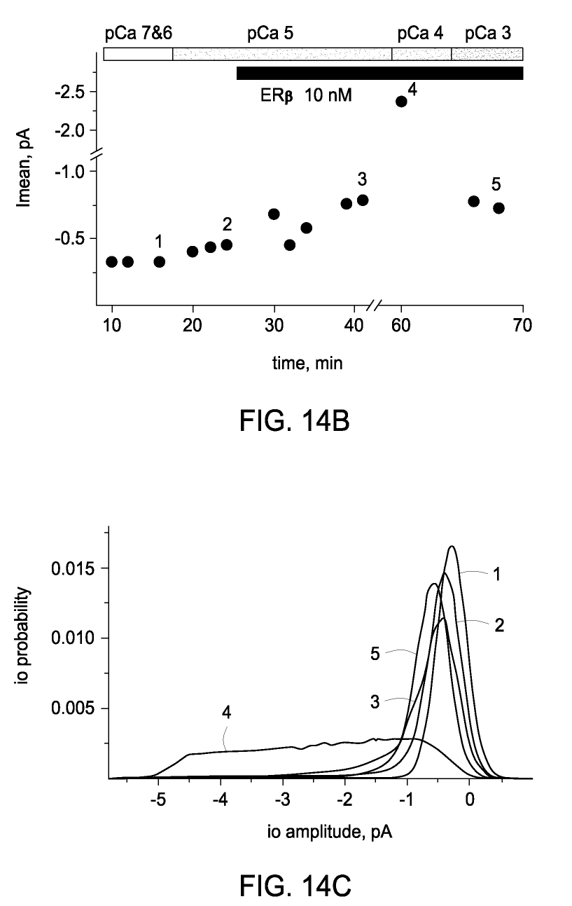

FIG. 15 shows that RyR2 and ERβ are co-localized in cytoplasmic compartments of the neuronal HT-22 cells. Immunostaining with mouse RyR2 (green) and ERβ (red) antibodies reveals the areas of co-localization of both proteins (yellow). Scale bars correspond to 20 μm.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

Chaban, V. V., A. J. Lakhter, and P. Micevych. 2004. A membrane estrogen receptor mediates intracellular calcium release in astrocytes. Endocrinology. 145:3788-95.
Chaban, V. V., and P. E. Micevych. 2005. Estrogen receptor-alpha mediates estradiol attenuation of ATP-induced Ca2+ signaling in mouse dorsal root ganglion neurons. J Neurosci Res. 81:31-7.
Chang, H. T., J. K. Huang, J. L. Wang, J. S. Cheng, K. C. Lee, Y. K. Lo, M. C. Lin, K. Y. Tang, and C. R. Jan. 2001. Tamoxifen-induced Ca2+ mobilization in bladder female transitional carcinoma cells. Arch Toxicol. 75:184-8.
Chang, H. T., J. K. Huang, J. L. Wang, J. S. Cheng, K. C. Lee, Y. K. Lo, C. P. Liu, K. J. Chou, W. C. Chen, W. Su, Y. P. Law, and C. R. Jan. 2002. Tamoxifen-induced increases in cytoplasmic free Ca2+ levels in human breast cancer cells. Breast Cancer Res Treat. 71:125-31.
Improta-Brears, T., A. R. Whorton, F. Codazzi, J. D. York, T. Meyer, and D. P. McDonnell. 1999. Estrogen-induced activation of mitogen-activated protein kinase requires mobilization of intracellular calcium. Proc Natl Acad Sci USA. 96:4686-91.
Jan, C. R., C. An-Jen, H. T. Chang, C. J. Roan, Y. C. Lu, B. P. Jiann, C. M. Ho, and J. K. Huang. 2003. The anti-breast cancer drug tamoxifen alters Ca2+ movement in Chinese hamster ovary (CHO-K1) cells. Arch Toxicol. 77:160-6.
Kim, J. K., and E. R. Levin. 2006. Estrogen signaling in the cardiovascular system. Nucl Recept Signal. 4:e013.
Levin, E. R. 2005. Integration of the extranuclear and nuclear actions of estrogen. Mol Endocrinol. 19:1951-9.
Mermelstein, P. G., J. B. Becker, and D. J. Surmeier. 1996. Estradiol reduces calcium currents in rat neostriatal neurons via a membrane receptor. J. Neurosci. 16:595-604.
Russell, K. S., M. P. Haynes, D. Sinha, E. Clerisme, and J. R. Bender. 2000. Human vascular endothelial cells contain membrane binding sites for estradiol, which mediate rapid intracellular signaling. Proc Natl Acad Sci USA. 97:5930-5.
Vasudevan, N., and D. W. Pfaff. 2006. Membrane Initiated Actions of Estrogens in Neuroendocrinology: Emerging Principles. Endocr Rev.

What is claimed is:

1. A method of screening for a candidate substance with ryanodine receptor (RyR)-modulatory activity, the method comprising:
determining the ion-conducting ability and ability to change the concentration of the free cytoplasmic intracellular Ca²⁺ by the RyR, modulated by Estrogen receptor-β (ERβ), in cells or cell membranes expressing RyR and ERβ in combination with, or in the absence of an estrogen;
contacting the cells or cell membranes with a candidate substance capable of modulation of the interaction between RyR and ERβ; and
measuring the RyR mediated ion-conducting ability of the cells or cell membranes to change the concentration of the free cytoplasmic intracellular Ca²⁺ by the candidate substance, whereby the modulatory activity of the candidate substance on RyR/ERβ interaction is determined.

2. The method of claim 1, wherein the RyR-expressing cells are primary brain, cardiac and vascular tissues or primary cell cultures, cells transfected with a RyR receptor or cell lines that express the RyR receptor.

3. The method of claim 1, wherein the cell membranes comprise bilayer lipid membranes (BLM), or Ca²⁺ release liposomes, microsomes or isolated nuclei.

4. The method of claim 1, wherein amount of internalized ERβ is between 1 pM to 100 μM.

5. The method of claim 1, wherein the RyR ion-conducting ability and ability to influence the concentration of the free cytoplasmic intracellular Ca²⁺ are measured electrophysiologically, fluorescently or calorimetrically.

6. The method of claim 1, wherein the candidate substance is an estrogen (as estradiol, 17β-estradiol, estriol, estrone) or a functional derivative, precursor, prodrug, homologue, analogue or salt thereof.

7. The method of claim 1, wherein the candidate substance is an ERβ-specific binding agent selected from small molecules, peptides and proteins, and agents selected from a small molecule library.

8. The method of claim 1, wherein the candidate substance is not-internalizable.

9. The method of claim 1, wherein the candidate substance is an ERβ-specific binding agent delivered into the cell by gene transfer or protein delivery comprising at least a portion of the ERβ.

10. The method of claim 1, wherein the candidate substance is a plasmid, cosmid, artificial chromosome, viroid, virus and virus-like particles, nanoparticle and electrical, magnetic or chemical delivery reagents that deliver nucleic acids that express peptides or proteins comprising at least a portion of the ERβ into cells.

11. A method of treatment of cardiac or vascular dysfunction in a human or animal subject comprising administering or intracellular synthesis of an effective amount of a low dose of an ERβ, ERβ fragment or derivative, ERβ-specific binding agent, including estrogens (estradiol, 17β-estradiol, estriol and estrone) and other hormones acting through ERβ, for a time and under conditions sufficient for correction of cardiac and vascular contraction/relaxation to occur thereby rectifying said cardiac and vascular dysfunction or pathology.

12. The method of claim 11, wherein the amount of the ERβ-specific binding agent is modulated based on the effect of the ERβ-specific binding agent on the RyR obtained from the subject measured by RyR ion conducting ability, Ca²⁺-induced Ca²⁺ release (CICR) or both.

13. The method of claim 11, wherein the cardiac dysfunction is myocardial contractile failure, ischemic heart disease, systemic inflammatory states such as sepsis, cardiac hypertrophy (calcium overload), cardiomyopathy such as arrhythmogenic right ventricular dysplasia type-2 (ARVD2), and drug-induced cardiomyopathy, infarction, dysrhythmia, congestive heart failure, or heart attack.

14. A dosage form comprising a low dose estrogen or candidate substance sufficient to treat a cardiovascular disease, wherein the dosage form is adapted to provide intracellular content of an estrogen or candidate substance that modulate the ERβ receptor based on the level of membrane ryanodine receptor (RyR) activity measured as ion-conducting ability of the RyR or Ca²⁺-induced Ca²⁺ release (CICR) from the endoplasmic reticulum of the cardiac vascular or neuronal tissue or primary cell culture in vitro.

15. The dosage form of claim 14, wherein the cardiac or vascular dysfunction is myocardial contractile failure, ischemic heart disease, systemic inflammatory states such as sepsis, cardiac hypertrophy (calcium overload), cardiomyopathy such as arrhythmogenic right ventricular dysplasia type-2 (ARVD2), and drug-induced cardiomyopathy, infarction, dysrhythmia, congestive heart failure, or heart attack.

16. The dosage form of claim 14, wherein the estrogen or candidate substance comprises an ERβ-specific binding agent.

17. The dosage form of claim 14, wherein the estrogen is an estrogen (estradiol, 17β-estradiol, E2, estriol, estrone) or a functional derivative, precursor, prodrug, homologue, analogue or salt thereof at concentration range between 1 pM to 100 μM.

18. The dosage form of claim 14, wherein the dosage is adapted for patients suffering from a loss of estrogen that is caused iatrogenically, by ovariectomy, by menopause, or due to normal aging.

19. The dosage form of claim 14, wherein the low dose estrogen or candidate substance decreases intracellular calcium release from intracellular stores.

20. The dosage form of claim 14, wherein the low dose estrogen or candidate substance crosses the blood-brain barrier.

21. The dosage form of claim 14, wherein the low dose estrogen or candidate substance is dissolved in a lipophilic pharmacophor and is suitable for intravenous injection, parenteral administration or oral administration and is administered one or more times daily over a predetermined period.

22. A method of treatment of neuronal dysfunction in a human or animal subject comprising administering or intracellular synthesis of an effective amount of a low dose of an ERβ, ERβ fragment or derivative, ERβ-specific binding agent, including estrogen and other hormones acting through ERβ, for a time and under conditions sufficient for reduced neurodegeneration, increased generation, mobility or interconnectivity of the neurons and other brain cells to occur, thereby rectifying said neuronal or brain dysfunction or pathology.

23. The method of claim 22, wherein the neuronal or brain dysfunction is selected from the group consisting of schizophrenia, minimal brain dysfunction, mania, Alzheimer's disease, attention deficit disorder (ADD), obsessive-compulsive disorder (OCD), learning deficit, dysmnesia, agnosia, amnesia and apraxia, Parkinsonism and its iatrogenic forms, Huntington's disease, glaucoma, macular degeneration, retinitis pigmentosa and acute diseases of the central nervous system.

24. A dosage form comprising a low dose estrogen or candidate substance sufficient to treat a neuronal dysfunction, wherein the dosage form is adapted to provide intracellular content of an estrogen or candidate substance that modulate the ERβ receptor based on the level of membrane ryanodine receptor (RyR) activity measured as ion-conducting ability of the RyR or Ca²⁺-induced Ca²⁺ release (CICR) from the endoplasmic reticulum of the cardiac vascular or neuronal tissue or primary cell culture in vitro.

25. The dosage form of claim 24, wherein the neuronal or brain dysfunction is selected from the group consisting of schizophrenia, minimal brain dysfunction, mania, Alzheimer's disease, attention deficit disorder (ADD), obsessive-compulsive disorder (OCD), learning deficit, dysmnesia, agnosia, amnesia and apraxia, Parkinsonism and its iatrogenic forms, Huntington's disease, glaucoma, macular degeneration, retinitis pigmentosa and acute diseases of the central nervous system.
